(19) United States
(12) Patent Application Publication
(10) Pub. No.: US 2021/0102019 A1
(43) Pub. Date: Apr. 8, 2021

(54) HYDROPHILIC COPOLYMER AND MEDICAL DEVICE

(71) Applicant: TERUMO KABUSHIKI KAISHA, Tokyo (JP)

(72) Inventors: Narumi YOKOTE, Fujisawa (JP); Masanori KURAMOTO, Hiratsuka (JP)

(73) Assignee: TERUMO KABUSHIKI KAISHA, Tokyo (JP)

(21) Appl. No.: 17/126,978

(22) Filed: Dec. 18, 2020

Related U.S. Application Data
(63) Continuation of application No. PCT/JP2019/025155, filed on Jun. 25, 2019.

(30) Foreign Application Priority Data
Jun. 27, 2018 (JP) 2018-122005

(51) Int. Cl. C08F 216/36; C09D 129/12; C08F 220/56; C08F 220/38; C08F 216/18; C09D 129/02; C09D 133/26; A61L 29/08; A61L 31/10

(52) U.S. Cl. CPC C08F 216/36; C09D 129/12; C08F 220/56; C08F 220/382; A61L 31/10; C09D 129/02; C09D 133/26; A61L 29/085; C08F 216/18

(57) ABSTRACT

Disclosed is a hydrophilic copolymer including more than 50% by mol of a structural unit derived from a polymerizable monomer (A) from which a homopolymer having a lower critical solution temperature (LCST) is formed, a structural unit derived from a polymerizable monomer (B) having at least one group selected from the group consisting of a sulfonic acid group (−SO₃H), a sulfate group (−OSO₃H), a sulfurous acid group (−OSO₂H), and a group of salts thereof, and a structural unit derived from a polymerizable monomer (C) having a photoreactive group. Also disclosed is a medical device including the hydrophilic copolymer. The medical device exhibits high sliding property until it reaches a target lesion, and low sliding property after reaching the target lesion. Methods of making and using the hydrophilic copolymer and medical device are also described.

### CROSS-REFERENCES TO RELATED APPLICATIONS

This application is a continuation of International Application No. PCT/JP2019/025155 filed on Jun. 25, 2019, which claims the benefit of Japanese Application No. 2018-122005 filed on Jun. 27, 2018, the entire content of both of which is incorporated herein by reference.

### FIELD OF THE INVENTION

The present invention generally relates to a medical device including a hydrophilic copolymer and a coating layer containing the hydrophilic copolymer.

### BACKGROUND DISCUSSION

A medical device intended to be inserted into a living body, such as a catheter, is required to have high sliding property in order to reduce damage to a biological tissue and improve operability by an operator. Further, the medical device described above reaches a target lesion while being moved or rotated in a longitudinal direction, and in the process, friction with an inner wall of a living body organ often occurs, and thus it is required to be able to withstand a plurality of frictions. Therefore, such a medical device must exhibit high sliding property until it reaches the target lesion (that is, it has high initial sliding property and can maintain high sliding property even after a plurality of frictions). On the other hand, the medical device used in some intervention procedures is required to exhibit low sliding property after reaching the target lesion so that a position does not shift when performing the operation on the target lesion.

Japanese Patent No. 4198348 discloses a medical device having a coating layer containing a temperature-sensitive polymer and a reactive polymer having a photoreactive group, and discloses that the lubricating property of the medical device changes after reaching a target site.

### SUMMARY

However, according to the studies of the inventors of the present invention, it has been found that the coating layer disclosed in Japanese Patent No. 4198348 has low initial sliding property in a steady environment (25° C.), and the sliding property greatly fluctuates when the medical device is rubbed a plurality of times. Such a coating layer does not provide a medical device with the high sliding property needed to reach a target lesion and reduce tissue damage.

This disclosure provides examples of a medical device that exhibits high sliding property until it reaches a target lesion (that is, the medical device has high initial sliding property and can maintain high sliding property even after a plurality of frictions), and low sliding property after reaching the target lesion.

As a result of intensive studies to solve the above problems, the present inventors have found that a high sliding property is achieved by a hydrophilic copolymer including more than 50% by mol of a structural unit derived from a polymerizable monomer (A) from which a homopolymer having a lower critical solution temperature (LCST) is formed, a structural unit derived from a polymerizable monomer (B) having at least one group selected from the group consisting of a sulfonic acid group (—SO₃H), a sulfate group (—OSO₃H), a sulfurous acid group (—OSO₂H), and a group of salts thereof, and a structural unit derived from a polymerizable monomer (C) having a photoreactive group, and have completed the present invention.

According to another aspect, a medical device comprises: a base material layer that possesses a surface; and a coating layer which is formed on at least a part of the surface of the base material layer and contains a hydrophilic copolymer. The hydrophilic copolymer comprises more than 50% by mol of a structural unit derived from a polymerizable monomer (A) from which a homopolymer having a lower critical solution temperature is formed, a structural unit derived from a polymerizable monomer (B) having at least one group selected from the group consisting of a sulfonic acid group (−SO₃H), a sulfate group (—OSO₃H), a sulfurous acid group (—OSO₂H), and a group of salts thereof, and a structural unit derived from a polymerizable monomer (C) having a photoreactive group.

### BRIEF DESCRIPTION OF THE DRAWINGS

FIG. 1 is a partial cross-sectional view schematically illustrating a laminated structure of a surface of a typical embodiment of a medical device described herein (hereinafter, also simply referred to as a medical device).

FIG. 2 is a partial cross-sectional view schematically illustrating a configuration example having a different surface laminated structure as an application example of the embodiment of FIG. 1.

FIG. 3 is a schematic view of a friction measuring machine used in a sliding property test described in the Examples.

FIG. 4 is a graph illustrating a change in a test force (sliding resistance) when reciprocation for the sliding property test is performed 10 times in water at 25° C. for the coating layers of the Examples and Comparative Examples.

FIG. 5 is a graph illustrating a test force (sliding resistance) when the reciprocation for the sliding property test is performed once in water at 60° C. for the coating layers of the Examples and Comparative Examples.

### DETAILED DESCRIPTION

Set forth below with reference to the accompanying drawings is a detailed description of a medical device that exhibits high sliding property until it reaches a target lesion, and low sliding property after reaching the target lesion, a method for producing such a medical device, a hydrophilic copolymer, a method for producing such a hydrophilic copolymer, and a method for treating a target lesion using the medical device disclosed here, all representing non-limiting examples of the inventions disclosed here.

Further, as used herein, “X to Y” indicating a range includes X and Y and means “X or more and Y or less”. Unless otherwise specified, the operation and physical properties are measured under the conditions of room temperature (20° C. to 25° C.)/relative humidity of 40% to 60% RH.

As used herein, the term “(meth)acrylic” includes both acrylic and methacrylic. Thus, for example, the term “(meth)acrylic acid” includes both acrylic acid and methacrylic acid. Similarly, the term “(meth)acryloyl” includes both acryloyl and methacryloyl. Thus, for example, the term “(meth)acryloyl group” includes both an acryloyl group and a methacryloyl group.

In a case where a certain structural unit is described herein as being “derived” from a certain monomer, the structural unit is a divalent structural unit generated when the polymerizable unsaturated double bond (C═C) existing in monomer corresponding to the structural unit becomes a single bond (—C—C—).

### Hydrophilic Copolymer

According to embodiments described herein, a hydrophilic copolymer includes more than 50% by mol of a structural unit derived from a polymerizable monomer (A) (hereinafter, referred to as monomer A) from which a homopolymer having a lower critical solution temperature (LCST) is formed, a structural unit derived from the polymerizable monomer (B) (hereinafter, referred to as monomer B) having at least one group selected from the group consisting of a sulfonic acid group (—SO₃H), a sulfate group (—OSO₃H), a sulfurous acid group (—OSO₂H), and a group of salts thereof, and a structural unit derived from a polymerizable monomer (C) (hereinafter, referred to as monomer C) having a photoreactive group.

A coating layer containing the hydrophilic copolymer according to one embodiment of the present invention has high initial sliding property in a steady environment (25° C.) and can maintain high sliding property even after a plurality of frictions. On the other hand, when the coating layer containing the hydrophilic copolymer is heated, the sliding property is significantly reduced. Therefore, a medical device having the coating layer on the surface thereof can exhibit high sliding property until it reaches a target lesion, and low sliding property after reaching the target lesion, by controlling temperature.

According to the study by the present inventors, it has been found that the coating layer disclosed in Japanese Patent No. 4198348 has low initial (first reciprocating sliding) sliding property in a steady environment (25° C.) (refer to Comparative Example 4-1 described later). Therefore, as a result of intensive studies on the configuration of the coating layer, the present inventors have found that the initial sliding property in a steady environment (25° C.) is dramatically improved by using the above-described monomer B as a raw material. The sulfonic acid group (—SO₃H), the sulfate group (—OSO₃H), the sulfurous acid group (—OSO₂H), or a group of salts thereof contained in monomer B have a higher hydration energy than other substituents, and thus are easily anionized and easily hydrated with the surrounding water. Therefore, it is considered that the coating layer containing the structural unit derived from monomer B has improved sliding property.

Further, the structural unit derived from monomer A has a property that a homopolymer has LCST (that is, it is temperature sensitive), in other words, it changes from hydrophilic to hydrophobic as the temperature rises. Therefore, it is considered that when the coating layer containing the structural unit is heated, the moisture contained in the coating layer is released, the coating layer shrinks, the surface becomes rough, and thereby the sliding property is deteriorated.

Further, the photoreactive group contained in the structural unit derived from monomer C generates reactive species by irradiation with active energy rays, and extracts a hydrogen atom from a hydrocarbon group existing in a base material (base material layer) or a copolymer so as to form a covalent bond. Therefore, the coating layer containing the structural unit is firmly fixed on a base material. Moreover, since the coating layer itself is also crosslinked, the strength of the coating layer is improved. Therefore, it is considered that the coating layer to be formed is less likely to be broken by friction (friction resistance is improved).

However, although the coating layer disclosed in Japanese Patent No. 4198348 contains a photoreactive group, it has been found that the sliding property fluctuates significantly with a plurality of frictions (refer to Comparative Example 4-1 described later). As the reason for this, the inventors presume that, since the coating layer disclosed in Japanese Patent No. 4198348 includes a mixture of polymers, a temperature-sensitive polymer is easily eluted and, as a result, an upper part of the coating layer is not firmly fixed to the base material and is separated by friction. Based on this presumption, in a case of an embodiment of a copolymer of monomer A, monomer B, and monomer C, it has been found that the coating layer to be formed can maintain high sliding property even after a plurality of frictions (e.g., ten sliding reciprocations). With such an embodiment, the elution of the components derived from monomer A and monomer B is suppressed. As a result, it is considered that the coating layer is firmly fixed to the base material over the entire coating layer, and it is less likely to be separated due to the friction.

The above-described mechanism is a presumption, and the present invention is not limited by such a presumption.

Hereinafter, each polymerizable monomer constituting the hydrophilic copolymer will be described.

### Polymerizable Monomer

### Monomer A

As monomer A, it is preferable that the homopolymer has a lower critical solution temperature (LCST) of 30° C. to 70° C., and examples thereof include N-isopropyl acrylamide (NIPAAm) (LCST of about 32° C.), N-vinyl isopropyl acrylamide (LCST of about 39° C.), N-vinyl-n-propyl acrylamide (LCST of about 32° C.), vinyl methyl ether (LCST of about 34° C.), 2-ethyl-2-oxazoline (LCST of about 65° C.), and 2-isopropyl-2-oxazoline (LCST of about 38° C.). In the above, the numerical values in parentheses represent the LCST of the homopolymer. By using such a monomer A, the lower critical solution temperature (LCST) of the obtained hydrophilic copolymer can be within a desired range (e.g., 40° C. to 70° C.). Among them, monomer A is particularly preferably N-isopropyl acrylamide (NIPAAm).

Monomer A may be used alone or two or more kinds thereof may be used in combination. Further, as monomer A, either a synthetic product or a commercially available product may be used. As a commercially available product, it can be obtained from, for example, Sigma-Aldrich Co. LLC or other commercial distributors.

In the hydrophilic copolymer, the content of the structural units derived from monomer A is more than 50% by mol when the total of the structural units derived from all the monomers is 100% by mol. In a case where the content is 50% by mol or less, the sliding property of the coating layer to be formed is not deteriorated to a desired range even when heated (refer to Comparative Example 5-2 described later). Therefore, even after the medical device reaches the target lesion and is heat-treated, the sliding property remains high, which may cause misalignment. Therefore, the lower limit of the content is preferably 60% by mol or more, more preferably 70% by mol or more, even more preferably 80% by mol or more, particularly preferably 85% by mol or more, and most preferably 90% by mol or more. In addition, to further improve the sliding property in the steady environment (25° C.) at the initial stage or/and after a plurality of frictions, and allowing the medical device to reach the target lesion more smoothly, the upper limit of the content is preferably 98% by mol or less, more preferably 96% by mol or less, and most preferably 94% by mol or less. The content is substantially the same as the ratio of the charged amount (mol) of monomer A to the total charged amount (mol) of the monomers in producing the polymer.

### Monomer B

Monomer B is a polymerizable monomer having at least one group selected from the group consisting of a sulfonic acid group (—SO₃H), a sulfate group (—OSO₃H), a sulfurous acid group (—OSO₂H), and a group of salts thereof. The salt is not particularly limited, and examples thereof include a sodium salt, a potassium salt, and an ammonium salt. In addition to the above groups, monomer B preferably has an ethylenically unsaturated group such as a (meth)acryloyl group, a vinyl group, or an allyl group.

Among them, from the viewpoint of further improving the sliding property in a steady environment (25° C.), monomer B is preferably a compound represented by any of the following Formulae (2), (3) or (4), and more preferably a compound represented by the following Formula (2).

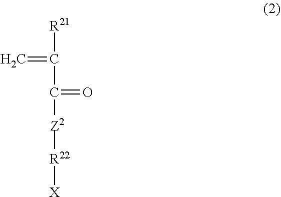

In the above Formula (2), R²¹ is a hydrogen atom or a methyl group, and preferably a hydrogen atom. Further, Z² is an oxygen atom (—O—) or —NH—, and preferably —NH—.

In the above Formula (2), from the viewpoint of further improving the sliding property in a steady environment (25° C.), R²² is a straight chain or branched chain alkylene group having 1 to 20 carbon atoms, preferably a straight chain or branched chain alkylene group having 1 to 12 carbon atoms, more preferably a straight chain or branched chain alkylene group having 1 to 8 carbon atoms, even more preferably a straight chain or branched chain alkylene group having 1 to 6 carbon atoms, particularly preferably a branched chain alkylene group having 3 to 5 carbon atoms. The branched chain alkylene group having 3 to 5 carbon atoms is a group represented by —CH(CH₃)—CH₂—, —C(CH₃)₂—CH₂—, —CH(CH₃)—CH(CH₃)—, —C(CH₃)₂—CH₂—CH₂—, —CH(CH₃)—CH(CH₃)—CH₂—, —CH(CH₃)—CH₂—CH(CH₃)—, —CH₂—C(CH₃)₂—CH₂—, and —C(CH₃)₂—CH(CH₃)— (here, connection order of the above groups in the above Formula (2) is not particularly limited), among them, the group represented by —C(CH₃)₂—CH₂— is particularly preferable.

In the above Formula (2), X is a group selected from the group consisting of a sulfonic acid group (—SO₃H), a sulfate group (—OSO₃H), a sulfurous acid group (—OSO₂H), and a group of salts thereof, and since the acid dissociation degree is high (that is, it is easily anionized) and the sliding property in a steady environment (25° C.) can be further improved, it is preferably a group selected from the group consisting of a sulfonic acid group, a sulfate group, and a group of salts thereof, and is more preferably a sulfonic acid group or a group of salt thereof in terms of availability of a monomer.

As an example of the compound represented by the above Formula (2), 2-(meth)acrylamide-2-methyl-1-propanesulfonic acid, 1-[(meth)acryloyloxymethyl]-1-propanesulfonic acid, 2-[(meth)acryloyloxy]-2-propanesulfonic acid, 3-[(meth)acryloyloxy]-1-methyl-1-propanesulfonic acid, 2-Sulfoethyl (meth)acrylate, 3-sulfopropyl (meth)acrylate, and salts thereof and the like. The salt is not particularly limited, and examples thereof include a sodium salt, a potassium salt, and an ammonium salt. These compounds may be used alone or in combination of two or more. Among them, 2-acrylamide-2-methyl-1-propanesulfonic acid (AMPS) and salts thereof are preferable.

The compound represented by the above Formula (2) may be either a synthetic product or a commercially available product, and can be obtained from, for example, Tokyo Chemical Industry Co., Ltd. or other distributors as a commercially available product.

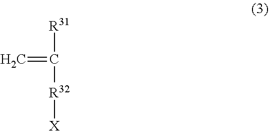

In the above Formula (3), R³¹ is a hydrogen atom or a methyl group.

In the above Formula (3), R³² is a single bond or a straight chain or branched chain alkylene group having 1 to 20 carbon atoms, preferably a single bond or a straight chain or branched chain alkylene group having 1 to 12 carbon atoms, more preferably a single bond or a straight chain or branched chain alkylene group having 1 to 8 carbon atoms, even more preferably a single bond or a straight chain or branched chain alkylene group having 1 to 4 carbon atoms, particularly preferably a single bond. Here, since the specific example of the alkylene group is the same as that represented by the above Formula (2), the description thereof will be omitted here.

In the above Formula (3), X is a group selected from the group consisting of a sulfonic acid group (—SO₃H), a sulfate group (—OSO₃H), a sulfurous acid group (—OSO₂H), and a group of salts thereof, and since the acid dissociation degree is high (that is, it is easily anionized) and the sliding property in a steady environment (25° C.) can be further improved, it is preferably a group selected from the group consisting of a sulfonic acid group, a sulfate group, and a group of salts thereof, and is more preferably a sulfonic acid group or a group of salt thereof in terms of availability of a monomer.

Examples of the compound represented by the above Formula (3) include vinyl sulfonic acid, allyl sulfonic acid, metalryl sulfonic acid, 2-propen-1-sulfonic acid, 2-methyl-2-propen-1-sulfonic acid, and salts thereof. These compounds may be used alone or in combination of two or more.

The compound represented by the above Formula (3) may be either a synthetic product or a commercially available product, and can be obtained from, for example, Asahi Kasei Finechem Co., Ltd, Tokyo Chemical Industry Co., Ltd. or other distributors as a commercially available product (for example, 2-methyl-2-propen-1-sulfonate sodium salt).

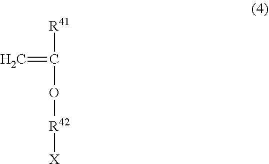

In the above Formula (4), R⁴¹ is a hydrogen atom or a methyl group.

In the above Formula (4), R⁴² is a straight chain or branched chain alkylene group having 1 to 20 carbon atoms, preferably a straight chain or branched chain alkylene group having 1 to 12 carbon atoms, more preferably a straight chain or branched chain alkylene group having 1 to 8 carbon atoms, even more preferably a straight chain or branched chain alkylene group having 1 to 6 carbon atoms. Here, since the specific example of the alkylene group is the same as that represented by the above Formula (2), the description thereof will be omitted here.

In the above Formula (4), X is a group selected from the group consisting of a sulfonic acid group (—SO₃H), a sulfate group (—OSO₃H), a sulfurous acid group (—OSO₂H), and a group of salts thereof, and since the acid dissociation degree is high (that is, it is easily anionized) and the sliding property in a steady environment (25° C.) can be further improved, it is preferably a group selected from the group consisting of a sulfonic acid group, a sulfate group, and a group of salts thereof, and is more preferably a sulfonic acid group or a group of salt thereof in terms of availability of a monomer.

Examples of the compound represented by the above Formula (4) include 2-sulfoxyethyl vinyl ether, 3-sulfoxy-n-propyl vinyl ether, and salts thereof. These compounds may be used alone or in combination of two or more.

As the compound represented by the above Formula (4), either a synthetic product or a commercially available product may be used.

In the hydrophilic copolymer, the lower limit of the content of the structural unit derived from monomer B is preferably 0.5% by mol or more, more preferably 1% by mol or more, even more preferably 2% by mol or more, and particularly preferably 4% by mol or more, when the total of the structural units derived from all the monomers is 100% by mol. If the content is equal to or more than the above lower limit, since the sliding property in the initial and/or after a plurality of frictions in a steady environment (25° C.) is increased, the medical device can reach the target lesion more smoothly. On the other hand, an upper limit of the content is preferably 30% by mol or less, more preferably 20% by mol or less, even more preferably 10% by mol or less, and particularly preferably 8% by mol or less. If the content is equal to or less than the upper limit, the sliding property can be significantly reduced by heating. Therefore, by heat-treating the medical device after reaching the target lesion, misalignment can be satisfactorily prevented. Note that, the content is substantially the same as the ratio of the charged amount (mol) of monomer B to the total charged amount (mol) of all monomers in producing the polymer.

Further, in the hydrophilic copolymer, the molar ratio of the structural unit derived from monomer A and the structural unit derived from monomer B (monomer A: monomer B) is preferably 70:30 to 99.5:0.5, more preferably 80:20 to 99:1, even more preferably 85:15 to 98:2, and particularly preferably 90:10 to 97:3. If the lower limit of the ratio range is 70:30 or more, since the sliding property of the coating layer is sufficiently reduced by heating, the misalignment can be prevented by heat-treating the medical device after reaching the target lesion. If the upper limit of the ratio range is 99.5:0.5 or less, since the sliding property in the initial and/or after a plurality of frictions in a steady environment (25° C.) becomes higher, the medical device can reach the target lesion more smoothly.

### Monomer C

Monomer C is a polymerizable monomer having a photoreactive group. Here, the photoreactive group refers to a group capable of generating reactive species such as radicals, nitrenes, and carbenes by irradiation with active energy rays and reaction with the base material layer so as to form a chemical bond. In addition to the photoreactive group, monomer C preferably has an ethylenically unsaturated group such as a (meth)acryloyl group, a vinyl group, or an allyl group.

Examples of the photoreactive group include an azide group, a diazo group, a diazirine group, a ketone group, and a quinone group.

Examples of the azide group include an aryl azide group such as phenyl azide and 4-fluoro-3-nitrophenyl azide; an acyl azide group such as benzoyl azide and p-methyl benzoyl azide; an azidoformate group such as ethyl azide formate and phenyl azideformate; a sulfonyl azide group such as benzene sulfonyl azide; and a phosphoryl azide group such as diphenyl phosphoryl azide and diethyl phosphoryl azide.

As the diazo group, groups derived from diazoalkane such as diazomethane and diphenyl diazomethane; diazoketone such as diazoacetophenone, 1-trifluoromethyl-1-diazo-2-pentanone; diazoacetate such as t-butyl diazoacetate and phenyl diazoacetate; α-diazoacetoacetate such as t-butyl-α-diazoacetoacetate; and the like.

Examples of the diazirine group include a group derived from 3-trifluoromethyl-3-phenyldiadirin and the like.

Examples of the ketone group include groups having a structure such as acetophenone, benzophenone, anthrone, xanthine, and thioxanthone.

Examples of the quinone group include groups derived from anthraquinone and the like.

These photoreactive groups are appropriately selected according to the type of the base material layer of the medical device and the like. For example, in a case where the base material layer is formed of a polyolefin resin such as polyethylene resin, a polyamide resin, a polyurethane resin, a polyester resin, or the like, it is preferably a ketone group or a phenyl azide group, and more preferably a group having a benzophenone structure (benzophenone group) from the viewpoint of easy availability of a monomer. That is, in one embodiment, monomer C has a benzophenone structure.

Examples of monomer C include 2-azidoethyl (meth)acrylate, 2-azidopropyl (meth)acrylate, 3-azidopropyl (meth)acrylate, 4-azidobutyl (meth)acrylate, 4-(meth)acryloyloxy benzophenone, 4-(meth)acryloyloxyethoxy benzophenone, 4-(meth)acryloyloxy-4′-methoxy benzophenone, 4-(meth)acryloyloxyethoxy-4′-methoxy benzophenone, 4-(meth)acryloyloxy-4′-bromobenzophenone, 4-(meth)acryloyloxyethoxy-4′-bromobenzophenone, 4-styryl methoxy benzophenone, and 4-(meth)acryloyloxythioxanthone. Among them, 4-(meth)acryloyloxy benzophenone is preferable.

Monomer C may be either a synthetic product or a commercially available product, and can be obtained from MRC Unitech Co., Ltd. or the like as a commercially available product.

In the hydrophilic copolymer, the lower limit of the content of the structural unit derived from monomer C is preferably 0.1% by mol or more, more preferably 0.2% by mol or more, even more preferably 0.5% by mol or more, and particularly preferably 1% by mol or more, when the total of the structural units derived from all the monomers is 100% by mol. If the content is equal to or more than the above lower limit, the hydrophilic copolymer can be sufficiently bonded to the base material (base material layer), so that the coating layer to be formed can be firmly fixed by the base material. Moreover, since the coating layer itself is also crosslinked, the strength of the coating layer is improved. Therefore, the coating layer to be formed is less likely to be broken by friction (friction resistance is improved). In addition, the upper limit of the content is preferably 40% by mol or less, more preferably 20% by mol or less, even more preferably 10% by mol or less, particularly preferably 5% by mol or less, and most preferably 3% by mol or less. If the content is equal to or less than the upper limit, the copolymer can be easily synthesized. In addition, since a sufficient amount of other monomers (monomers A and B) can be present, the coating layer to be formed has high sliding property in a steady environment (25° C.), and the sliding property is significantly reduced by heating. Therefore, it is advantageous in achieving both smooth arrival of the medical device at the target lesion and prevention of misalignment at the target lesion. Note that, the content is substantially the same as the ratio of the charged amount (mol) of monomer C to the total charged amount (mol) of the entire monomer in producing the polymer.

As long as the high sliding property is not impaired, the hydrophilic copolymer may contain a structural unit derived from a polymerizable monomer other than the above-described monomer A, monomer B, and monomer C (hereinafter, also referred to as “other monomers”). In the hydrophilic copolymer, the content of the structural unit derived from the other monomers is preferably less than 10% by mol, more preferably less than 5% by mol, and even more preferably less than 1% by mol, with respect to 100% by mol of the total of the structural units derived from all the monomers (lower limit: 0% by mol). Preferably, the hydrophilic copolymer includes monomer A, monomer B, and monomer C. Note that, the content is substantially the same as the ratio of the charged amount (mol) of the other monomers to the total charged amount (mol) of the entire monomer in producing the polymer.

A terminal of the hydrophilic copolymer is not particularly limited and is appropriately defined depending on the type of raw material to be used. Generally, it is a hydrogen atom. The structure of the copolymer is also not particularly limited, and may be a random copolymer, an alternating copolymer, a periodic copolymer, or a block copolymer. From the viewpoint of maintaining high sliding property even after a plurality of frictions, a random copolymer is preferable.

### Physical Properties of Hydrophilic Copolymer

### Lower Critical Solution Temperature (LCST)

The lower limit of the lower critical solution temperature (LCST) of the hydrophilic copolymer is preferably 40° C. or higher, more preferably 45° C. or higher, and even more preferably 50° C. or higher. When the temperature is 40° C. or higher, when the coating layer containing the copolymer is introduced into the body, the sliding property is not significantly deteriorated due to the influence of body temperature. In other words, even if the coating layer is introduced into the body, the high sliding property can be exhibited unless the heat treatment is intentionally performed. On the other hand, the upper limit of the LCST of the hydrophilic copolymer is preferably 70° C. or lower, more preferably 65° C. or lower, and even more preferably 60° C. or lower. If the temperature is 70° C. or lower, the sliding property of the coating layer is reduced by gentle heat treatment, so that there is little adverse effect on the subject such as degeneration of blood components. Therefore, the hydrophilic copolymer according to the embodiment has a lower critical solution temperature (LCST) of 40° C. to 70° C. In the present specification, the LCST of the hydrophilic copolymer is measured by the following method.

### Measuring Method of LCST

The hydrophilic copolymer is dissolved in methanol to a concentration of 10% by weight to prepare a coating liquid. Next, a nylon elastomer sheet (12.5 mm×100 mm) is dipped in the coating liquid and pulled up at a speed of 15 mm/sec. Then, the nylon elastomer sheet is dried at room temperature (25° C.) for one hour to remove the solvent. Next, a nylon elastomer sheet is irradiated with UV having a wavelength of 365 nm and a lamp power of 1 kW until the integrated light intensity is 500 mJ/cm² to obtain a sample. As the UV irradiation device, UVC-1212/1MNLC3-AA04 (high pressure mercury lamp) manufactured by Ushio, Inc. is used.

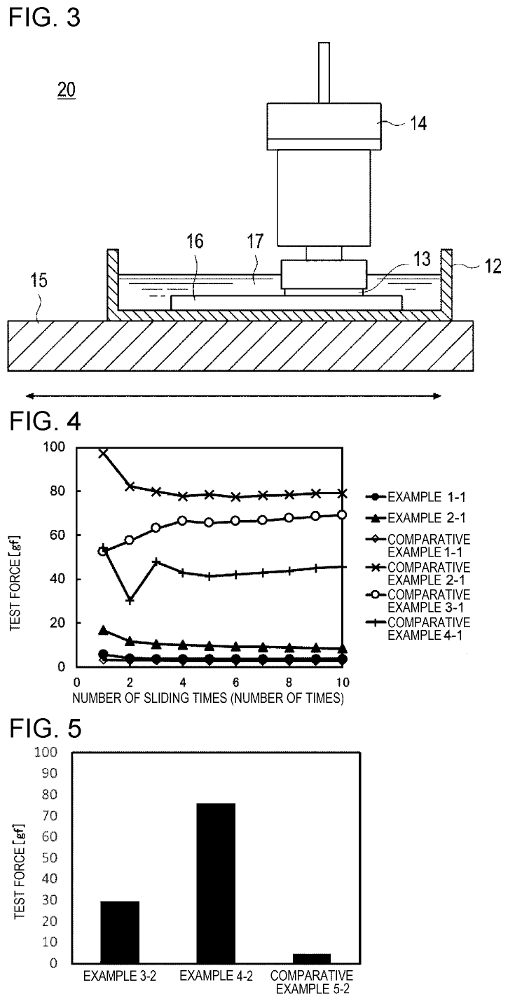

Next, the sliding property of the obtained sample is evaluated according to the following method using a friction measuring machine (Handy Tribomaster TL201 manufactured by Trinity-Lab Inc.) 20 illustrated in FIG. 3. Specifically, the sample 16 is fixed in a petri dish 12, immersed in water 17 at a predetermined temperature at a height at which the entire sample 16 is immersed, and allowed to stand for 10 seconds. The petri dish 12 is placed on a moving table 15 of the friction measuring machine 20 illustrated in FIG. 3. A silicon terminal (φ10 mm, R1 mm) 13 is brought into contact with the sheet, and a load 14 of 50 g is applied on the terminal. With the setting of a sliding distance of 20 mm and a sliding speed of 16.7 mm/sec, the sliding resistance (gf) when the moving table 15 is horizontally reciprocated once is measured.

In the measuring method described above, the temperature of the water 17 in which the sample 16 is immersed is changed from 25° C. at intervals of 5° C., the sliding resistance (gf) during the first reciprocating outward path at each temperature is measured, and the lowest temperature among the temperatures at which the value exceeds 20 gf is defined as the lower critical solution temperature (LCST) of the hydrophilic copolymer.

### Molecular Weight

A weight average molecular weight of the hydrophilic copolymer is preferably 1,000 to 500,000, more preferably 2,000 to 200,000, even more preferably 5,000 to 100,000, particularly preferably 10,000 to 50,000, and most preferably 20,000 to 40,000. In the present specification, as the weight average molecular weight, a value measured by gel permeation chromatography (GPC) using polystyrene as a standard substance is adopted.

### Method of Producing Hydrophilic Copolymer

The method of producing the hydrophilic copolymer is not particularly limited, and known polymerization methods such as radical polymerization, anionic polymerization, and cationic polymerization can be adopted, and radical polymerization which is easy to produce is preferably used.

As the polymerization method, a method of polymerizing the above-described monomer A, monomer B, monomer C, and if necessary, other monomers by stirring and heating together with a polymerization initiator in a polymerization solvent is adopted.

The polymerization temperature is not particularly limited, and is preferably 25° C. to 100° C., and more preferably 30° C. to 80° C. The polymerization time is also not particularly limited, and is preferably 30 minutes to 24 hours, and more preferably 1 to 5 hours.

A polymerization solvent is preferably an aqueous solvent such as water; alcohols such as methanol, ethanol, propanol, n-butanol, 2,2,2-trifluoroethanol; and polyhydric alcohols such as ethylene glycol, diethylene glycol, propylene glycol, and dipropylene glycol. From the viewpoint of dissolving the raw materials used for the polymerization, these may be used alone or two or more kinds thereof may be used in combination.

The concentration of the polymerizable monomer is not particular limited, and the total solid content (g) of each polymerizable monomer with respect to the polymerization solvent (mL) is preferably 0.05 to 1 g/mL, and more preferably 0.1 to 0.5 g/mL. Further, the preferable range of the ratio of the charged amount (mol) of each monomer to the total charged amount (mol) of all the monomers is as described above.

The reaction solution containing the polymerizable monomer may be subjected to a degassing treatment before the polymerization initiator is added. The degassing treatment may be performed by bubbling the reaction solution with an inert gas such as nitrogen gas or argon gas for about 0.5 to 5 hours. During the degassing treatment, the reaction solution may be heated to about 30° C. to 100° C.

For the production of polymers, known polymerization initiators in the related art can be used, and the present invention is not particularly limited. For example, azobispolymerization initiators such as 2,2′-azobisisobutyronitrile, 2,2′-azobis(4-methoxy-2,4-dimethyl valeronitrile), 4,4′-azobis(4-cyanovaleric acid), 2,2′-azobis(2,4-dimethyl valeronitrile); and a redox-based polymerization initiator that combines a reducing agent such as sodium sulfite, sodium hydrogen sulfite, and ascorbic acid to an oxidizing agent such as persulfates such as potassium persulfate (KPS), sodium persulfate, and ammonium persulfate, and peroxides such as hydrogen peroxide, t-butyl peroxide, and methyl ethyl ketone peroxide can be used.

The blending amount of the polymerization initiator is preferably 0.01% to 10% by mol, more preferably 0.1° A to 5% by mol, with respect to the total amount (mol) of the polymerizable monomers.

Further, if necessary, a chain transfer agent, a polymerization rate adjusting agent, a surfactant, and other additives may be appropriately used in the polymerization.

The environment (atmosphere) in which the polymerization reaction is performed is not particularly limited, and the polymerization reaction can be performed in an atmospheric atmosphere (i.e., under atmospheric conditions), an atmosphere of an inert gas such as nitrogen gas or argon gas, or the like. Further, the reaction solution may be stirred during the polymerization reaction.

The copolymer may precipitate during the polymerization reaction. The copolymer after polymerization can be purified by a general purification method such as a reprecipitation method, a dialysis method, an ultrafiltration method, or an extraction method.

The purified copolymer can be dried by any method such as freeze-drying, vacuum-drying, spray-drying, or heat-drying. From the viewpoint of having a small effect on the physical properties of the polymer, freeze-drying or vacuum-drying is preferable.

The ratio of the structural units derived from each polymerizable monomer in the obtained copolymer can be checked by analyzing the peak intensity of the group contained in each structural unit using known means such as NMR and IR.

The content of unreacted monomers contained in the obtained copolymer is preferably 0.01% by weight or less with respect to the total amount of the copolymer. The smaller the content of the unreacted monomers, the more preferable (lower limit: 0% by weight). The content of residual monomers can be measured by known means such as high performance liquid chromatography.

### Medical Device

The present invention also provides a medical device having a base material layer and a coating layer formed on at least a portion of the surface of the base material layer and containing the above-described hydrophilic copolymer.

Hereinafter, preferable embodiments of the medical device will be described with reference to the attached drawings.

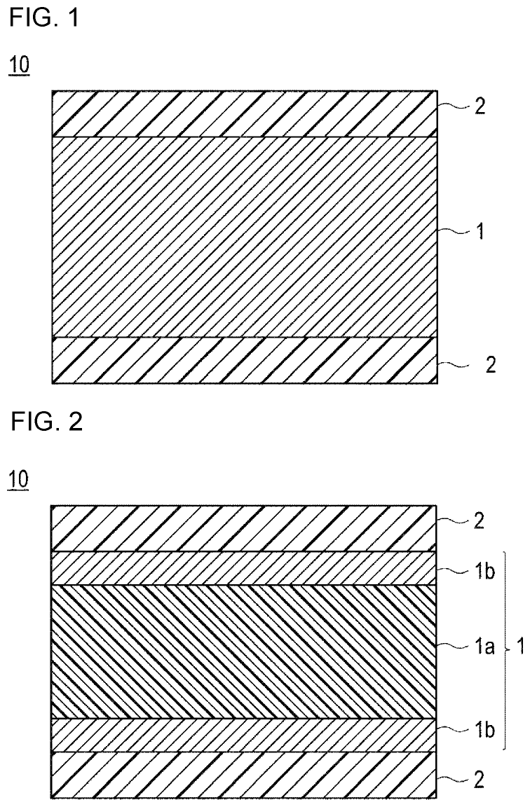

FIG. 1 is a partial cross-sectional view schematically illustrating a laminated structure of a surface of a typical embodiment of a medical device. FIG. 2 is a partial cross-sectional view schematically illustrating a configuration example having a different surface laminated structure as an application example of the embodiment. In FIGS. 1 and 2, reference numeral 1 represents a base material layer, reference numeral 1a represents a base material layer core portion, reference numeral 1b represents a base material surface layer, reference numeral 2 represents a coating layer, and reference numeral 10 represents a medical device.

As illustrated in FIGS. 1 and 2, the medical device 10 of the present embodiment includes a base material layer 1 and a coating layer 2 containing a hydrophilic copolymer fixed on at least a portion of the base material layer 1. The drawings illustrate an example of fixation on the entire surface (entire surface) of the base material layer 1 in the drawings. The coating layer 2 is bonded to the base material layer 1 via a photoreactive group of the hydrophilic copolymer.

Hereinafter, each configuration of the medical device of the present embodiment will be described.

### Base Material Layer (Base Material)

The base material layer used in the present embodiment may be formed of any material as long as it can react with the photoreactive group contained in the above hydrophilic copolymer so as to form a chemical bond. Specifically, examples of the material forming the base material layer 1 include a metal material, a polymer material, and ceramics. Here, the base material layer 1 is, as illustrated in FIG. 1, the entire (all) base material layer 1 may be formed of any of the above materials, or as illustrated in FIG. 2, the surface of the base material layer core portion 1a formed of any of the above materials may be coated (coated) with any other materials than the above described materials by an appropriate method to have a structure in which the base material surface layer 1b is formed. Examples of the latter case include a structure in which the surface of the base material layer core portion 1a formed of a resin material or the like is coated with a metal material by an appropriate method (plating, metal deposition, sputtering, or the like of known methods in the related art) to form the base material surface layer 1b; and a structure in which the surface of the base material layer core portion 1a formed of a hard reinforcing material such as a metal material or a ceramic material is coated with a polymer material that is more flexible than the reinforcing material such as a metal material by an appropriate method (dipping, spraying, coating/printing, or the like of known methods in the related art) or the reinforcing material of the base material layer core portion 1a and the polymer material of the base material surface layer 1b are composited (appropriately treated) so as to form the base material surface layer 1b. Therefore, the base material layer core portion 1a may be a multi-layer structure in which different materials are laminated in multiple layers, or a structure (composite) in which members formed of different materials may be connected to each part of the medical device. Further, another middle layer (not shown) may be formed between the base material layer core portion 1a and the base material surface layer 1b. Further, the base material surface layer 1b may be a multi-layer structure in which different materials are laminated in multiple layers, or a structure (composite) in which members formed of different materials may be connected to each part of the medical device.

Among the materials forming the base material layer 1, the metal material is not particularly limited, metal materials commonly used in medical devices such as balloons, catheters, guide wires, micro balloons, micro catheters, micro guide wires, stent delivery catheters, ablation catheters are used. Specific examples thereof include various stainless steels (SUS) such as SUS304, SUS316, SUS316L, SUS420J2, and SUS630, gold, platinum, silver, copper, nickel, cobalt, titanium, iron, aluminum, and tin, or various alloys such a nickel-titanium (Ni—Ti) alloy, a nickel-cobalt (Ni—Co) alloy, a cobalt-chromium (Co—Cr) alloy, and a zinc-tungsten (Zn—W) alloy. These may be used alone or two or more kinds thereof may be used in combination. As the metal material, the most suitable metal material as a base material layer for balloons, catheters, guide wires, micro balloons, micro catheter, micro guide wires, stent delivery catheters, ablation catheters, and the like, which are intended for use may be appropriately selected.

In addition, among the materials forming the base material layer 1, the polymer material is not particularly limited, and polymer materials (such as elastomers) commonly used in medical devices such as balloons, catheters, guide wires, micro balloons, micro catheters, micro guide wires, stent delivery catheters, and ablation catheters are used. Specific examples thereof include a polyamide resin, a polyamide elastomer (such as a nylon elastomer), polyethylene such as straight chain low density polyethylene (LLDPE), low density polyethylene (LDPE), and high density polyethylene (HDPE), a polyolefin resin such as polypropylene, a polyester resin such as polyethylene terephthalate, a polyester elastomer, a styrol resin such as polystyrene, a cyclic polyolefin resin, a modified polyolefin resin, an epoxy resin, a urethane resin, a dialyl phthalate resin (allyl resin), a polycarbonate resin, a fluororesin, an amino resin (urea resin, melamine resin, or benzoguanamine resin), an acrylic resin, a polyacetal resin, a vinyl acetate resin, a phenolic resin, a vinyl chloride resin, a silicone resin (silicon resin), a polyether resin, and a polyimide resin. These may be used alone or two or more kinds thereof may be used in combination. As the polymer material, the most suitable polymer material as a base material layer for balloons, catheters, guide wires, micro balloons, micro catheter, micro guide wires, stent delivery catheters, ablation catheters, and the like, which are intended for use may be appropriately selected.

The shape of the base material layer is not particularly limited, and is appropriately selected depending on the usage mode such as sheet shape, wire shape, and tubular shape.

### Method of Producing the Medical Device

The method of producing a medical device according to the present invention (method of forming a coating layer on a base material layer) is not particularly limited except that the above hydrophilic copolymer is used, and a known method can be applied in the same manner or modified as appropriate. For example, a method in which the hydrophilic copolymer according to the present invention is dissolved in a solvent to prepare a coating liquid, and the coating liquid is coated on the base material layer of the medical device is preferable.

### Coating Step

In the above method, the solvent used to dissolve the hydrophilic copolymer can be appropriately selected, and examples thereof include methanol, ethanol, n-propanol, isopropanol, and the like.

The concentration of the hydrophilic copolymer in the coating liquid is not particularly limited, and is preferably 0.01% to 50% by weight, more preferably 0.05% to 40% by weight, and even more preferably 0.1° A to 30% by weight. Within such a range, the coating property of the coating liquid is excellent. Further, a uniform coating layer having a desired thickness can be easily obtained by one coating, which is preferable in terms of production efficiency. In a case where the concentration of the hydrophilic copolymer is less than 0.01% by weight, it may not be possible to fix a sufficient amount of the hydrophilic copolymer on the surface of the base material layer. Further, in a case where the concentration of the hydrophilic copolymer exceeds 50% by weight, the viscosity of the coating liquid becomes excessively high, and a coating layer having a uniform thickness may not be obtained. However, even if the concentration of the hydrophilic copolymer deviates from the above range, the coating solution containing the hydrophilic copolymer can be sufficiently used as long as it does not affect the action and effect of the present invention.

Before applying the coating liquid, the surface of the base material layer may be treated in advance by an ultraviolet irradiation treatment, a plasma treatment, a corona discharge treatment, a flame treatment, an oxidation treatment, a silane coupling treatment, a phosphoric acid coupling treatment or the like. In a case where the solvent of the coating liquid is only water, it is difficult to apply the water to the surface of the hydrophobic base material layer, but the surface of the base material layer is made hydrophilic by the plasma treatment on the surface of the base material layer. As a result, the wettability of the coating liquid to the surface of the base material layer is improved, and a uniform coating layer can be formed. Further, by performing the above treatment on the surface of the base material layer that does not have a C—H bond such as a metal or a fluororesin, it is possible to form a covalent bond with the photoreactive group of the hydrophilic copolymer.

The method of coating the surface of the base material layer with the coating liquid is not particularly limited. Known methods in the related art, such as a coating/printing method, a dip method (dipping method, dip coating method), a spray method, a spin coating method, and a mixed solution impregnated sponge coating method can be applied. Among them, the dip method (dipping method, dip coating method) is preferable.

In a case where the coating layer is formed on the inner surface of the medical device having a small inner diameter such as a catheter, the base material layer may be immersed in the coating liquid to reduce the pressure inside the system to defoam. By defoaming under reduced pressure, the solution can be quickly permeated into the small and narrow inner surface, and the formation of the coating layer can be promoted.

In addition, in a case where the coating layer is formed only on a portion of the base material layer, by immersing only a portion of the base material layer in the coating liquid and coating a portion of the base material layer with the coating liquid, a coating layer can be formed on a desired surface portion of the base material layer.

In a case where it is difficult to immerse only a portion of the base material layer in the coating liquid, after protecting (coating or the like) the surface part of the base material layer, which does not need to form the coating layer in advance, with an appropriate member or material that can be attached/detached, the base material layer is immersed into the coating liquid and the base material layer is coated with the coating liquid, the protective member (material) on the surface of the base material layer that does not need to form the coating layer is detached, and then, a coating layer can be formed on a desired surface portion of the base material layer by reacting with a heating operation or the like. However, the present invention is not limited to these forming methods, and a coating layer can be formed by appropriately using a known method in the related art. For example, in a case where it is difficult to immerse only a portion of the base material layer in a mixed solution, instead of the dip method, other coating methods may be used. For example, a method of coating a predetermined surface part of a medical device with a coating liquid using a coating device such as a spray device, a bar coater, a die coater, a reverse coater, a comma coater, a gravure coater, a spray coater, or a doctor knife may be applied.

In a case where it is necessary for both the outer surface and the inner surface of the cylindrical device to have a coating layer in terms of the structure of the medical device, the dip method (dipping method) is preferably used because both the outer surface and the inner surface can be coated at one time.

Drying the Coating Film:

It is preferable to immerse the base material layer in the coating liquid containing the hydrophilic copolymer of the present invention as described above, remove the base material layer from the coating liquid, and then dry the coating film. The drying conditions are not particularly limited as long as the solvent of the coating liquid can be removed, and a warm air treatment may be performed using a dryer or the like, or natural drying may be performed. Further, the pressure condition at the time of drying is not limited at all, and it can be performed under normal pressure (atmospheric pressure), or under pressure or reduced pressure. As the drying means (device), for example, an oven, a vacuum dryer, or the like can be used, but in the case of natural drying, drying means (device) is not particularly required.

Fixing the Coating Layer on the Surface of the Base Material Layer:

The coating film after the drying step is irradiated with active energy rays. As a result, the photoreactive group of the hydrophilic copolymer in the coating film is activated, and a covalent bond is formed between the copolymer and the base material layer or between the copolymers.

The formation of a covalent bond between the hydrophilic copolymer and the base material layer will be described below by taking a combination of a hydrophilic copolymer having a benzophenone structure as a photoreactive group and a polyethylene base material layer as an example. In a case where the hydrophilic copolymer contains a photoreactive group having a benzophenone structure, irradiation with ultraviolet rays generates two radicals in the photoreactive group. One of these radicals extracts a hydrogen atom from the polyethylene base material layer, and instead, one radical is generated on the polyethylene base material layer. After that, the remaining radicals in the photoreactive group and the radicals on the polyethylene base material layer are bonded to form a covalent bond between the hydrophilic copolymer and the polyethylene base material layer. By such a mechanism, the coating layer containing the hydrophilic copolymer of the present invention is firmly fixed on the surface of the base material layer.

Examples of the active energy ray include ultraviolet rays, electron beams, and gamma rays, and the ultraviolet rays or electron beams are preferable, and the ultraviolet rays are more preferable in consideration of the influence on the human body. In a case where the ultraviolet rays are used, a wavelength at which the photoreactive group can be activated can be appropriately selected as the irradiation wavelength. The irradiation intensity of ultraviolet rays is not particularly limited, and is preferably 1 to 5000 mW/cm². The integrated amount of ultraviolet rays is also not particularly limited, and is preferably 50 to 5000 mJ/cm², and more preferably 100 to 1000 mJ/cm². Examples of the device for irradiating ultraviolet rays include a high-pressure mercury lamp, a low-pressure mercury lamp, a metal halide lamp, a xenon lamp, and a halogen lamp.

After performing the above active energy ray irradiation, the surface of the base material layer may be washed with a solvent (for example, a solvent used for preparing the coating liquid) to remove the unreacted hydrophilic copolymer.

The fixation of the coating film (coating layer) on the base material layer can be checked by using known analytical means such as FT-IR and XPS. For example, it can be checked by performing FT-IR measurement before and after irradiation with the active energy ray and comparing the ratio of the bond peak formed by the active energy ray irradiation with the invariant bond peak.

By the above method, a coating layer containing the hydrophilic copolymer of the present invention is formed on the surface of the medical device according to the present invention. The coating layer has high initial sliding property in a steady environment (25° C.), and can maintain the high sliding property even after a plurality of frictions. On the other hand, when the coating layer is heated, the sliding property is significantly reduced. Therefore, a medical device having the coating layer on the surface thereof can exhibit high sliding property until it reaches the target lesion, and low sliding property after reaching the target lesion, by controlling temperature.

In the coating layer of the medical device according to the present invention, the sliding resistance in the steady environment (25° C.) is preferably 20 gf or less, more preferably 15 gf or less, even more preferably 10 gf or less, and particularly preferably 5 gf or less (lower limit: 0 gf). If the above upper limit or less can be maintained even after a plurality of frictions, the medical device can smoothly reach the target lesion or can be smoothly recovered from the target lesion.

On the other hand, in the coating layer of the medical device according to the present invention, the sliding resistance at 60° C. is preferably 25 gf or more, more preferably 30 gf or more, even more preferably 40 gf or more, and particularly preferably 50 gf or more. If it is equal to or more than the above lower limit, after the medical device reaches the target lesion, the sliding can be lowered by the heat treatment, and an accurate treatment can be performed without causing the misalignment. Note that, the upper limit of the value is not particularly limited, and is, for example, 200 gf or less.

The sliding resistances of the coating layer at 25° C. and 60° C. are measured using a friction measuring machine (Handy Tribomaster TL201 manufactured by Trinity-Lab Inc.) 20 illustrated in FIG. 3. Specifically, as illustrated in FIG. 3, the sample 16 having the coating layer on the upper surface is fixed in the petri dish 12, immersed in water 17 at 25° C. or 60° C. at a height at which the entire sample 16 is immersed, and allowed to stand for 10 seconds. The petri dish 12 is placed on the moving table 15 of the friction measuring machine 20. A silicon terminal (φ10 mm, R1 mm) 13 is brought into contact with the sheet, and a load 14 of 50 g is applied on the terminal. With the setting of a sliding distance of 20 mm and a sliding speed of 16.7 mm/sec, the sliding resistance (gf) when the moving table 15 is horizontally reciprocated ten times is measured.

The sliding property of the medical device obtained as described above can be controlled by temperature. Therefore, another embodiment of the present invention is a method of using the medical device in which the medical device is heated after reaching the target lesion (target site).

The method of heating the medical device is not particularly limited, and examples thereof include a method of connecting a fluid supply source to a medical device and supplying a heated fluid (for example, physiological salt solution) from the fluid supply source to the inside of the medical device. See Japanese Patent Application Publication No. 2015-97547 and corresponding to U.S. Patent Application Publication No. 2015/018873, and a method of connecting an energy supply source to a medical device and supplying electric energy from the energy supply source to the medical device (refer to JP-A-2017-195910 or the like).

The lower limit of the heating temperature of the medical device is preferably 40° C. or higher, and more preferably 50° C. or higher from the viewpoint of shortening the working time. On the other hand, the upper limit of the heating temperature is preferably 70° C. or lower, more preferably 65° C. or lower, and even more preferably 60° C. or lower in consideration of safety to the living body. In addition, the heating time of the medical device varies depending on the heating temperature and the like, and is preferably within 1 minute.

After the treatment is performed on the target lesion, the coating layer is naturally cooled to about body temperature by stopping the heating of the medical device. As a result, the sliding property of the coating layer is restored, and the medical device can be smoothly recovered from the target lesion. At this time, the medical device may be intentionally cooled for the purpose of shortening the working time. Examples of the method of cooling the medical device include a method in which a refrigerant supply source is connected to the medical device and the refrigerant is supplied from the refrigerant supply source to the inside of the medical device.

The medical device according to the present invention is not particularly limited as long as it can be heated by the above method or the like after reaching the target lesion, and examples thereof include balloons, catheters, guide wires, micro balloons, micro catheter, micro guide wires, stent delivery catheters, and ablation catheters. More specifically, the following medical devices are exemplified:

(a) catheters that are orally or nasally inserted or indwelled in the digestive organs such as gastric tube catheters, feeding catheters, and cervical nutrition tubes;

(b) catheters that are orally or nasally inserted or indwelled in the airways or trachea such as oxygen catheters, oxygen canulass, tubes and cuffs of endotracheal tubes, tubes and cuffs of tracheostomy tubes, and endotracheal suction catheters;

(c) urethral catheters, urinary catheters, urethral balloon catheters, and catheters that are inserted or indwelled in the urethra or ureter such as balloons;

(d) catheters inserted or indwelled in various body cavities, organs, and groups, such as a suction catheter, a drainage catheter, and a rectal catheter;

(e) catheters that are inserted or indwelled in a blood vessel, such as an indwelling needle, a IVH catheter, a thermodilution catheter, an angiography catheter, a micro catheter, a balloon catheter for vasodilatation, a micro balloon catheter, a stent delivery catheter, a dilator or introducer, and/or guide wires for these catheters, micro guide wires, stylets, and the like;

(f) artificial trachea, artificial bronchus, and the like.

(g) medical devices (e.g., artificial lungs, artificial hearts, artificial kidneys, or the like) for extracorporeal circulation treatment and circuits thereof; and

(h) ablation catheters.

### EXAMPLES

The present disclosure will be described in more detail with reference to the following Examples. However, these embodiments are examples only and do not limit the present disclosure.

Each part and % in each Example is based on weight. Hereinafter, all the conditions left at room temperature, unless otherwise specified, are 23° C./55% RH.

### Production of Hydrophilic Copolymer

### Production Example 1

1.06 g (9.4 mmol) of N-isopropyl acrylamide (NIPAAm) produced by Tokyo Chemical Industry Co., Ltd., 0.183 g (0.4 mmol) of 2-acrylamide-2-methyl-1-propanesulfonic acid sodium salt (AMPS (Na)), and 0.053 g (0.2 mmol) of 4-methacryloyloxy benzophenone (MBP) produced by MRC Unitech Co., Ltd were dissolved in 10 mL of a 2,2,2-trifluoroethanol/water (9/1 v/v) mixed solvent to prepare a reaction solution. Next, this reaction solution was placed in a 30 mL eggplant-shaped flask, oxygen was removed by sufficient nitrogen bubbling, and 28 mg (0.100 mmol) of a polymerization initiator (V-501 produced by Wako Pure Chemical Industries, Ltd.) was added, and the mixture was quickly sealed and polymerized in a water bath at 80° C. for 2 hours. Next, it was subjected to precipitation in ether, and the supernatant was removed by decantation, followed by drying under reduced pressure, to obtain a copolymer. The obtained copolymer was a random compound and had a weight average molecular weight of about 30,000. Note that, the weight average molecular weight is a value measured by gel permeation chromatography (GPC) using polystyrene as a standard substance.

### Production Example 2

1.02 g (9.0 mmol) of N-isopropyl acrylamide (NIPAAm) produced by Tokyo Chemical Industry Co., Ltd., 0.367 g (0.8 mmol) of 2-acrylamide-2-methyl-1-propanesulfonic acid sodium salt (AMPS (Na)), and 0.053 g (0.2 mmol) of 4-methacryloyloxy benzophenone (MBP) produced by MRC Unitech Co., Ltd were dissolved in 10 mL of a 2,2,2-trifluoroethanol/water (9/1 v/v) mixed solvent to prepare a reaction solution. Next, this reaction solution was placed in a 30 mL eggplant-shaped flask, oxygen was removed by sufficient nitrogen bubbling, and 28 mg (0.100 mmol) of a polymerization initiator (V-501 produced by Wako Pure Chemical Industries, Ltd.) was added, and the mixture was quickly sealed and polymerized in a water bath at 80° C. for 2 hours. Next, it was subjected to precipitation in ether, and the supernatant was removed by decantation, followed by drying under reduced pressure, to obtain a copolymer. The obtained copolymer was a random compound and had a weight average molecular weight of about 30,000.

### Production Example 3

0.566 g (5.0 mmol) of N-isopropyl acrylamide (NIPAAm) produced by Tokyo Chemical Industry Co., Ltd., 2.20 g (4.8 mmol) of 2-acrylamide-2-methyl-1-propanesulfonic acid sodium salt (AMPS (Na)), and 0.053 g (0.2 mmol) of 4-methacryloyloxy benzophenone (MBP) produced by MRC Unitech Co., Ltd were dissolved in 10 mL of a 2,2,2-trifluoroethanol/water (9/1 v/v) mixed solvent to prepare a reaction solution. Next, this reaction solution was placed in a 30 mL eggplant-shaped flask, oxygen was removed by sufficient nitrogen bubbling, and 28 mg (0.100 mmol) of a polymerization initiator (V-501 produced by Wako Pure Chemical Industries, Ltd.) was added, and the mixture was quickly sealed and polymerized in a water bath at 80° C. for 2 hours. Next, it was subjected to precipitation in acetone, and the supernatant was removed by decantation, followed by drying under reduced pressure, to obtain a copolymer. The obtained copolymer was a random compound and had a weight average molecular weight of about 30,000.

### Production Example 4

1.11 g (9.8 mmol) of N-isopropyl acrylamide (NIPAAm) produced by Tokyo Chemical Industry Co., Ltd., and 0.053 g (0.2 mmol) of 4-methacryloyloxy benzophenone (MBP) produced by MRC Unitech Co., Ltd were dissolved in 10 mL of a 2,2,2-trifluoroethanol/water (9/1 v/v) mixed solvent to prepare a reaction solution. Next, this reaction solution was placed in a 30 mL eggplant-shaped flask, oxygen was removed by sufficient nitrogen bubbling, and 28 mg (0.100 mmol) of a polymerization initiator (V-501 produced by Wako Pure Chemical Industries, Ltd.) was added, and the mixture was quickly sealed and polymerized in a water bath at 80° C. for 2 hours. Next, it was subjected to precipitation in ether, and the supernatant was removed by decantation, followed by drying under reduced pressure, to obtain a copolymer. The obtained copolymer was a random compound and had a weight average molecular weight of about 30,000.

### Production Example 5

1.09 g (9.8 mmol) of 1-vinyl-2-pyrrolidone (VP) produced by Tokyo Chemical Industry Co., Ltd., and 0.053 g (0.2 mmol) of 4-methacryloyloxy benzophenone (MBP) produced by MRC Unitech Co., Ltd were dissolved in 10 mL of a 2,2,2-trifluoroethanol/water (9/1 v/v) mixed solvent to prepare a reaction solution. Next, this reaction solution was placed in a 30 mL eggplant-shaped flask, oxygen was removed by sufficient nitrogen bubbling, and 28 mg (0.100 mmol) of a polymerization initiator (V-501 produced by Wako Pure Chemical Industries, Ltd.) was added, and the mixture was quickly sealed and polymerized in a water bath at 80° C. for 2 hours. Next, it was subjected to precipitation in ether, and the supernatant was removed by decantation, followed by drying under reduced pressure, to obtain a copolymer (corresponding to the reactive polymer of Japanese Patent No. 4198348). The obtained copolymer was a random compound and had a weight average molecular weight of about 30,000.

### Production Example 6

4.13 g (48.5 mmol) of N-vinyl acetamide (NAV) produced by Tokyo Chemical Industry Co., Ltd., and 20.8 g (242 mmol) of vinyl acetate (VA) produced by Tokyo Chemical Industry Co., Ltd. were dissolved in 37.5 mL of ethanol to prepare a reaction solution. Next, this reaction solution was placed in a 100 mL eggplant-shaped flask, oxygen was removed by sufficient nitrogen bubbling, and 0.4 g (2.44 mmol) of a polymerization initiator (AIBN produced by Wako Pure Chemical Industries, Ltd.) was added, and the mixture was quickly sealed and polymerized in a water bath at 60° C. for 2 hours. Next, it was subjected to precipitation in ether, and the supernatant was removed by decantation, followed by drying under reduced pressure, to obtain a copolymer (corresponding to the temperature sensitive polymer of Japanese Patent No. 4198348). The obtained copolymer was a random compound and had a weight average molecular weight of about 30,000.

TABLE 1

| | Structural unit ratio (% by mol) | | | | | |
|---|---|---|---|---|---|---|
| | NIPAAm | VP | NAV | VA | AMPS(Na) | MBP |
| Production | 94 | — | — | — | 4 | 2 |
| Example 1 | | | | | | |
| Production | 90 | — | — | — | 8 | 2 |
| Example 2 | | | | | | |
| Production | 50 | — | — | — | 48 | 2 |
| Example 3 | | | | | | |
| Production | 98 | — | — | — | — | 2 |
| Example 4 | | | | | | |
| Production | — | 98 | — | — | — | 2 |
| Example 5 | | | | | | |
| Production | — | — | 17 | 83 | — | — |
| Example 6 | | | | | | |

In Table 1 above, each abbreviation is as follows:

NIPAAm: N-isopropyl acrylamide (corresponding to monomer A)

VP: 1-vinyl-2-pyrrolidone

NAV: N-vinyl acetamide

VA: Vinyl acetate

AMPS (Na): 2-acrylamide-2-methyl-1-propanesulfonic acid sodium salt (corresponding to monomer B)

MBP: 4-methacryloyloxy benzophenone (corresponding to monomer C)

### Sliding Property Test at Water Temperature 25° C.

### Example 1-1

A copolymer (corresponding to the hydrophilic copolymer according to the present invention) obtained by Production Example 1 was dissolved in methanol to a concentration of 10% by weight to prepare a coating liquid. Next, a nylon elastomer sheet (12.5 mm×100 mm) was dipped in the coating liquid and pulled up at a speed of 15 mm/sec. The nylon elastomer sheet was then dried at room temperature for one hour to remove the solvent. Next, the nylon elastomer sheet was irradiated with UV having a wavelength of 365 nm and a lamp power of 1 kW until the integrated light intensity was 500 mJ/cm2 to obtain a sample. As the UV irradiation device, UVC-1212/1MNLC3-AA04 (high pressure mercury lamp) manufactured by Ushio, Inc. was used.

Next, the sliding property of the obtained sample was evaluated according to the following method using a friction measuring machine (Handy Tribomaster TL201 manufactured by Trinity-Lab Inc.) 20 illustrated in FIG. 3.

Sample 16 was fixed in a petri dish 12, immersed in water 17 at 25° C. at a height at which the entire sample 16 was immersed, and allowed to stand for 10 seconds. The petri dish 12 was placed on a moving table 15 of the friction measuring machine 20 illustrated in FIG. 3. A silicon terminal (φ10 mm, R1 mm) 13 was brought into contact with the sheet, and a load 14 of 50 g was applied on the terminal. With the setting of a sliding distance of 20 mm and a sliding speed of 16.7 mm/sec, the sliding resistance (gf) when the moving table 15 was horizontally reciprocated ten times is measured. The change in the sliding resistance for 10 repeated slidings was evaluated by averaging the sliding resistance during the outward path from the first to tenth reciprocation and plotting them as a test force on a graph.

### Example 2-1

A sample was prepared and the sliding resistance was measured in the same manner as in Example 1-1 except that the copolymer obtained in Production Example 2 was used instead of the copolymer obtained in Production Example 1.

### Comparative Example 1-1

A sample was prepared and the sliding resistance was measured in the same manner as in Example 1-1 except that the copolymer obtained in Production Example 3 was used instead of the copolymer obtained in Production Example 1.

### Comparative Example 2-1

A sample was prepared and the sliding resistance was measured in the same manner as in Example 1-1 except that the copolymer obtained in Production Example 4 was used instead of the copolymer obtained in Production Example 1 and acetone was used instead of methanol as a coating solvent.

### Comparative Example 3-1

A sample was prepared and the sliding resistance was measured in the same manner as in Example 1-1 except that 0.12 g of the copolymer obtained in Production Example 5 and 1.4 g of poly (N-isopropyl acrylamide) (PNIPAAm) produced by Sigma-Aldrich Co., Ltd. were dissolved in 25 mL of ethanol/water (4/1 v/v) to prepare the coating liquid.

### Comparative Example 4-1

A sample was prepared and the sliding resistance was measured in the same manner as in Example 1-1 except that 0.12 g of the copolymer obtained in Production Example 5 and 1.4 g of the copolymer obtained in Production Example 6 were dissolved in 15 mL of ethanol/water (2/1 v/v) to prepare the coating liquid.

FIG. 4 illustrates the results of the sliding property test at a water temperature of 25° C. The samples of Example 1-1, Example 2-1, and Comparative Example 1-1 illustrate a sliding resistance of 20 gf or less through the first to tenth reciprocation at 25° C., and high sliding property at the initial stage and after a plurality of frictions.

On the other hand, the samples of Comparative Example 2-1, Comparative Example 3-1, and Comparative Example 4-1 had a sliding resistance exceeding 20 gf at the initial stage (first reciprocation sliding). It is presumed that this is due to the absence of a structural unit derived from the monomer B, which is a slidable component. Further, regarding the samples of Comparative Example 3-1 and Comparative Example 4-1, the sliding resistance was significantly disturbed in the first to fourth reciprocation, and a significant increase in the sliding resistance was observed in the fifth to tenth reciprocation. Since the coating layer of these samples is formed of a mixture of polymers, it is considered that PNIPAAm having no photoreactive group or the copolymer obtained in Production Example 6 was eluted from the coating layer.

### LCST Measurement of Hydrophilic Copolymer

The sliding resistance (gf) at each temperature was measured in the same manner as in Examples 1-1 and 1-2, except that the temperature of water 17 was changed to 30° C., 35° C., 40° C., 45° C., 50° C., 55° C., 60° C., 65° C. or 70° C. The lowest temperature among the temperatures at which the sliding resistance in the first reciprocation exceeded 20 gf was defined as the lower critical solution temperature (LCST) of the hydrophilic copolymer.

As a result of the above test, the LCST of the hydrophilic copolymers obtained in Production Example 1 and Production Example 2 was in the range of 40° C. to 70° C.

### Sliding Property Test at Water Temperature 60° C.

### Example 3-2

A copolymer (corresponding to the hydrophilic copolymer according to the present invention) obtained by Production Example 1 was dissolved in methanol to a concentration of 10% by weight to prepare a coating liquid. Next, a nylon elastomer sheet (12.5 mm×100 mm) was dipped in the coating liquid and pulled up at a speed of 15 mm/sec. The nylon elastomer sheet was then dried at room temperature for one hour to remove the solvent. Next, the nylon elastomer sheet is irradiated with UV having a wavelength of 365 nm and a lamp power of 1 kW until the integrated light intensity is 500 mJ/cm2. As the UV irradiation device, UVC-1212/1MNLC3-AA04 (high pressure mercury lamp) manufactured by Ushio, Inc. was used.

Next, the sliding property of the obtained sample was evaluated according to the following method using a friction measuring machine (Handy Tribomaster TL201 manufactured by Trinity-Lab Inc.) 20 illustrated in FIG. 3.

That is, the sample 16 was fixed in a petri dish 12, immersed in water 17 at 60° C. at a height at which the entire sample 16 was immersed, and allowed to stand for 10 seconds. The petri dish 12 was placed on a moving table 15 of the friction measuring machine 20 illustrated in FIG. 3. A silicon terminal (φ10 mm, R1 mm) 13 was brought into contact with the sheet, and a load 14 of 50 g was applied on the terminal. With the setting of a sliding distance of 20 mm and a sliding speed of 16.7 mm/sec, the sliding resistance (gf) when the moving table 15 was horizontally reciprocated once is measured. The change in the initial sliding resistance for temperature rise was evaluated by averaging the sliding resistance during the outward path at the first reciprocation and plotting them as a test force on a graph.

### Example 4-2

A sample was prepared and the sliding resistance was measured in the same manner as in Example 3-2 except that the copolymer obtained in Production Example 2 was used instead of the copolymer obtained in Production Example 1.

### Comparative Example 5-2

A sample was prepared and the sliding resistance was measured in the same manner as in Example 3-2 except that the copolymer obtained in Production Example 3 was used instead of the copolymer obtained in Production Example 1.

FIG. 5 illustrates the results of the sliding property test at a water temperature of 60° C. The samples of Example 3-2 and Example 4-2 indicate a sliding resistance of 25 gf or more at 60° C.

On the other hand, Comparative Example 5-2 indicates a low sliding resistance even at 60° C. Since the copolymer according to Production Example 3 contains few structural units derived from the monomer A, it is considered that the contribution of the structural unit derived from the monomer B was large and the sliding property was not deteriorated even when heated.

As apparent from the above results, the coating layer containing the hydrophilic copolymer according to the present invention has high initial (first reciprocation sliding) sliding property in a steady environment (25° C.) and can maintain high sliding property even after a plurality of frictions (ten sliding reciprocations). On the other hand, when the coating layer containing the hydrophilic copolymer according to the present invention is heated, the sliding property is significantly reduced. Therefore, a medical device having the coating layer on the surface thereof can exhibit high sliding property until it reaches the target lesion, and low sliding property after reaching the target lesion.

The detailed description above describes embodiments of a hydrophilic copolymer, a medical device including a hydrophilic copolymer, a coating layer containing the hydrophilic copolymer, a method for producing the hydrophilic copolymer, a method for producing a medical device including the hydrophilic copolymer, and a method of using such a medical device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical device comprising:
a base material layer that possesses a surface; and
a coating layer which is formed on at least a part of the surface of the base material layer and contains a hydrophilic copolymer, the hydrophilic copolymer comprising more than 50% by mol of a structural unit derived from a polymerizable monomer (A) from which a homopolymer having a lower critical solution temperature is formed, a structural unit derived from a polymerizable monomer (B) having at least one group selected from the group consisting of a sulfonic acid group (—SO₃H), a sulfate group (—OSO₃H), a sulfurous acid group (—OSO₂H), and a group of salts thereof, and a structural unit derived from a polymerizable monomer (C) having a photoreactive group.

2. The medical device according to claim 1, the medical device being a balloon, a catheter, a guide wire, a micro balloon, a micro catheter, a micro guide wire, a stent delivery catheter, or an ablation catheter.

3. The medical device according to claim 1, wherein the lower critical solution temperature is 40° C. to 70° C.

4. The medical device according to claim 1, wherein the polymerizable monomer (A) is at least one selected from the group consisting of N-isopropyl acrylamide, N-vinyl isopropyl acrylamide, N-vinyl-n-propyl acrylamide, vinyl methyl ether, 2-ethyl-2-oxazoline, and 2-isopropyl-2-oxazoline.

5. The medical device according to claim 1, wherein the polymerizable monomer (B) is represented by Formula (2), (3), or (4): 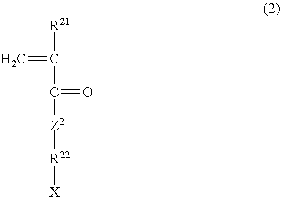
in Formula (2),
R²¹ is a hydrogen atom or a methyl group,
Z² is an oxygen atom or —NH—,
R²² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; 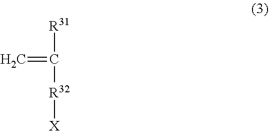
in Formula (3),
R³¹ is a hydrogen atom or a methyl group,
R³² is a single bond or a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; and 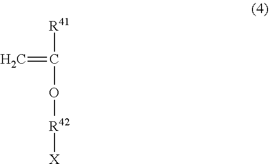
in Formula (4),
R⁴¹ is a hydrogen atom or a methyl group,
R⁴² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof.

6. The medical device according to claim 1, wherein the polymerizable monomer (C) has a benzophenone structure.

7. The medical device according to claim 1, wherein the base material layer is made of a metal material, a polymer material, or a ceramic.

8. A method of treating a lesion, the method comprising:
inserting a medical device into a living body;
advancing the medical device in the body toward the lesion so that a part of the medical device is positioned at the lesion;
increasing the temperature of the medical device after the part of the medical device is positioned at the lesion; and
reducing the temperature of the medical device prior to removing the medical device from the living body.

9. The method according to claim 8, the medical device being a balloon, a catheter, a guide wire, a micro balloon, a micro catheter, a micro guide wire, a stent delivery catheter, or an ablation catheter.

10. The method according to claim 8, the increasing the temperature of the medical device after the part of the medical device is positioned at the lesion comprises heating the medical device to a range of 40° C. to 70° C.

11. The method according to claim 8, the medical device comprising:
a base material layer that possesses a surface; and
a coating layer which is formed on at least a part of the surface of the base material layer and contains a hydrophilic copolymer, the hydrophilic copolymer comprising more than 50% by mol of a structural unit derived from a polymerizable monomer (A) from which a homopolymer having a lower critical solution temperature is formed, a structural unit derived from a polymerizable monomer (B) having at least one group selected from the group consisting of a sulfonic acid group (—SO₃H), a sulfate group (—OSO₃H), a sulfurous acid group (—OSO₂H), and a group of salts thereof, and a structural unit derived from a polymerizable monomer (C) having a photoreactive group.

12. The method according to claim 11, wherein the lower critical solution temperature is 40° C. to 70° C.

13. The method according to claim 11, wherein the polymerizable monomer (A) is at least one selected from the group consisting of N-isopropyl acrylamide, N-vinyl isopropyl acrylamide, N-vinyl-n-propyl acrylamide, vinyl methyl ether, 2-ethyl-2-oxazoline, and 2-isopropyl-2-oxazoline.

14. The method according to claim 11, wherein the polymerizable monomer (B) is represented by Formula (2), (3), or (4): 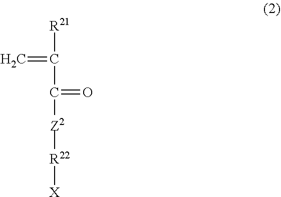
in Formula (2),
R²¹ is a hydrogen atom or a methyl group,
Z² is an oxygen atom or —NH—,
R²² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; 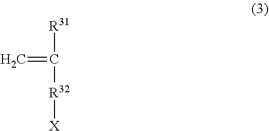
in Formula (3),
R³¹ is a hydrogen atom or a methyl group,
R³² is a single bond or a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; and 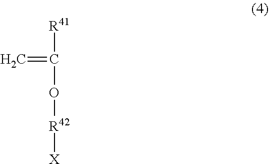
in Formula (4),
R⁴¹ is a hydrogen atom or a methyl group,
R⁴² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof.

15. A hydrophilic copolymer comprising:
more than 50% by mol of a structural unit derived from a polymerizable monomer (A) from which a homopolymer having a lower critical solution temperature is formed;
a structural unit derived from a polymerizable monomer (B) having at least one group selected from the group consisting of a sulfonic acid group (—SO₃H), a sulfate group (—OSO₃H), a sulfurous acid group (—OSO₂H), and a group of salts thereof; and
a structural unit derived from a polymerizable monomer (C) having a photoreactive group.

16. The hydrophilic copolymer according to claim 15, wherein the lower critical solution temperature is 40° C. to 70° C.

17. The hydrophilic copolymer according to claim 15, wherein the polymerizable monomer (A) is at least one selected from the group consisting of N-isopropyl acrylamide, N-vinyl isopropyl acrylamide, N-vinyl-n-propyl acrylamide, vinyl methyl ether, 2-ethyl-2-oxazoline, and 2-isopropyl-2-oxazoline.

18. The hydrophilic copolymer according to claim 15, wherein the polymerizable monomer (B) is represented by Formula (2), (3), or (4): 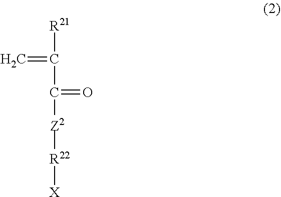
in Formula (2),
R²¹ is a hydrogen atom or a methyl group,
Z² is an oxygen atom or —NH—,
R²² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; 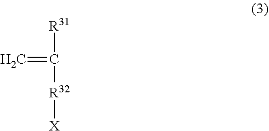
in Formula (3),
R³¹ is a hydrogen atom or a methyl group,
R³² is a single bond or a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof; and 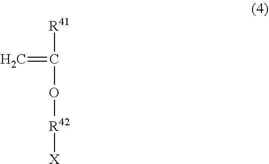
in Formula (4),
R⁴¹ is a hydrogen atom or a methyl group,
R⁴² is a straight chain or branched chain alkylene group having 1 to 20 carbon atoms, and
X is a group selected from the group consisting of a sulfonic acid group (—SO₃H), a sulfate group (—OSO₃H), a sulfurous acid group (—OSO₂H), and a group of salts thereof.

19. The hydrophilic copolymer according to claim 15, wherein the polymerizable monomer (C) has a benzophenone structure.

20. The hydrophilic copolymer according to claim 15, wherein the molar ratio of the structural unit derived from monomer A and the structural unit derived from monomer B (monomer A: monomer B) is 70:30 to 99.5:0.5.
